(19) United States
(12) Patent Application Publication
(10) Pub. No.: US 2024/0065612 A1
(43) Pub. Date: Feb. 29, 2024

(54) Systems And Methods For Early Breast Cancer Detection

(71) Applicant: Board of Regents, The University of Texas System, Austin, TX (US)

(72) Inventors: Babak Fahimi, Plano, TX (US); Carlos Caicedo-Narvaez, Richardson, TX (US); Yinan Li, San Jose, CA (US)

(21) Appl. No.: 17/766,464

(22) PCT Filed: Oct. 2, 2020
(86) PCT No.: PCT/US2020/054049

§ 371 Date: Apr. 4, 2022

Related U.S. Application Data
(60) Provisional application No. 62/911,129, filed on Oct. 4, 2019.

(51) Int. Cl. A61B 5/00; A61B 5/01; A61B 5/0507

(52) U.S. Cl. CPC A61B 5/4312; A61B 5/015; A61B 5/0507; A61B 5/6804

(57) ABSTRACT

Various examples are provided related to breast cancer detection. In one example, a method includes measuring a first temperature of a discrete location on the surface of the breast; emitting electromagnetic radiation into the breast at the discrete location and cause the tissue to generate heat; enabling the generated heat to reach the surface of the breast; measuring a second temperature of the discrete location on the surface of the breast; determining a temperature differential between the first and second temperatures; and determining whether or not cancerous tissue exists within the breast at the discrete location based upon the temperature differential. In another example, a system includes a plurality of electromagnetic emitters; a plurality of temperature sensors co-located with the electromagnetic emitters; and a control unit configured to activate the electromagnetic emitters and to receive temperature measurements taken by the temperature sensors.

### CROSS REFERENCE TO RELATED APPLICATIONS

This application claims priority to, and the benefit of, co-pending U.S. provisional application entitled “Systems and Methods for Early Breast Cancer Detection” having Ser. No. 62/911,129, filed Oct. 4, 2019, which is hereby incorporated by reference in its entirety.

### BACKGROUND

Second only to skin cancer, breast cancer is the most common cancer in women within the United States and the leading cause of cancer in Hispanic women. As with any cancer, early detection is critical in the treatment of breast cancer. Amongst all the breast cancer diagnostic techniques, mammography, a process involving micro-calcification analysis of the breast tissue using low-energy X-rays, is considered the gold standard. Mammography, however, often leads to false positives or over-diagnosis often due to detected abnormalities that meet the pathologic definition of cancer but that will never actually progress further. This over-diagnosis leads to unnecessary biopsies that can create discomfort or pain to the patient.

A more recent alternative to mammography is infrared thermography (IRT). With this technique, the temperature distribution across the surface of the breast is examined because localized high-temperature areas may result from blood vessel concentrations related to cancer growth. Although infrared thermography is a good non-invasive alternative to mammography, it is dependent on modeling to determine whether or not the heat abnormality is due to cancer or to the fact that the human breast is a fairly complex organ comprising various tissues and a labyrinth of blood vessels. In addition, infrared thermography can produce high rates of both false positives and negatives, and it is rarely covered by medical insurance, at least in part because of its known shortcomings.

A further problem with both mammography and infrared thermography is the duration of time that typically passes between screenings. In general, annual screening is recommended for women ages 45 to 54. However, a one-year window can be too long if the cancer happens to appear soon after an annual test is performed as the cancer then has a long time to grow before the next screening. As early detection is so critical, it would be better if a simple, accurate, and non-invasive screening process were available that could be performed with greater frequency, potentially in the comfort of one's own home.

### SUMMARY

Aspects of the present disclosure are related to breast cancer detection. In one aspect, among others, a method comprises measuring a first temperature of a discrete location on the surface of the breast; emitting electromagnetic radiation into the breast at the discrete location to excite any cancerous tissue below the surface and cause the cancerous tissue to generate heat; enabling the generated heat to reach the surface of the breast; measuring a second temperature of the discrete location on the surface of the breast; determining a temperature differential between the first and second temperatures; and determining whether or not cancerous tissue exists within the breast at the discrete location based upon the temperature differential. In one or more aspects, the method can further comprise determining a temperature differential in the same manner for multiple discrete locations on the surface of the breast. The temperature differential can be determined by sequentially exciting individual elements of an electromagnetic emitter array positioned over the breast. The method can comprise determining a three-dimensional temperature distribution in the breast based on an electro-thermal equivalent circuit of the breast, the electro-thermal equivalent circuit based upon bio-thermal tissue properties of the breast. A location of the cancerous tissue can be identified based upon the three-dimensional temperature distribution. Emitting electromagnetic radiation can comprise emitting radio frequency radiation. The radio frequency radiation can be in a frequency range from about 3 kHz to about 300 GHz. Determining whether or not cancerous tissue exists within the breast at the discrete location can comprise performing finite element analysis.

In various aspects, the method can comprise measuring a first temperature of a second discrete location on the surface of the breast; emitting the electromagnetic radiation into the breast at the second discrete location to excite any cancerous tissue below the surface and cause the cancerous tissue to generate heat; and measuring a second temperature of the second discrete location on the surface of the breast. The electromagnetic radiation can be emitted at the two discrete locations by two different electromagnetic emitters positioned over the breast. The two different electromagnetic emitters can be integrated in a garment worn over the breast. The electromagnetic radiation can be sequentially emitted at the two discrete locations by an electromagnetic emitter that is repositioned from the discrete location to the second discrete location.

In another aspect, a system for performing a breast examination to detect cancer can comprise a plurality of electromagnetic emitters provided within an array; a plurality of temperature sensors, one temperature sensor co-located with each electromagnetic emitter; a garment that supports the emitters and sensors configured for application to a breast of a patient; and a control unit configured to activate the electromagnetic emitters for the purpose of emitting electromagnetic radiation into the breast and to receive temperature measurements taken by the temperature sensors. In one or more aspects, the electromagnetic emitters can comprise patch antennas or multiple figure-eight elements. The temperature sensors can comprise thermistors.

In various aspects, the control unit can obtain a first temperature measurement prior to emitting the electromagnetic radiation into the breast by one of the plurality of electromagnetic emitters and a second temperature measurement after emitting the electromagnetic radiation into the breast, the temperature measurements obtained by the temperature sensor co-located with the one electromagnetic emitter. Presence of cancerous tissue can be determined based upon a temperature differential determined from the first and second temperature measurements. Temperature differentials can be determined by sequentially exciting individual elements of the plurality of electromagnetic emitters positioned over the breast. The plurality of electromagnetic emitters and the plurality of temperature sensors can be configured for application to one or both breasts of the patient.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a finite element analysis model of a breast, in accordance with various embodiments of the present disclosure.

FIGS. 2A-2C illustrate examples of patch antennas that can be used as electromagnetic emitters, in accordance with various embodiments of the present disclosure.

FIGS. 3A and 3B illustrate an example of electromagnetic emitter placement for detection of breast cancer, in accordance with various embodiments of the present disclosure.

FIG. 3C is a table illustrating properties of breast tissue, in accordance with various embodiments of the present disclosure.

FIG. 4 is a flow diagram illustrating an example of an examination to detect breast cancer, in accordance with various embodiments of the present disclosure.

FIG. 5A illustrates an example of electromagnetic emitter placement in a garment for detection of breast cancer, in accordance with various embodiments of the present disclosure.

FIGS. 5B and 5C illustrate another example of an electromagnetic emitter for detection of breast cancer, in accordance with various embodiments of the present disclosure.

FIG. 6 illustrate an example of measurement points for detection of breast cancer, in accordance with various embodiments of the present disclosure.

FIGS. 7A-C and 8A-8B illustrate examples of detection simulations, in accordance with various embodiments of the present disclosure.

FIGS. 9A-9C illustrate an example of a test setup for verification of breast cancer detection using electromagnetic emissions, in accordance with various embodiments of the present disclosure.

FIGS. 10A-10C illustrate examples of test measurements using the detection system, in accordance with various embodiments of the present disclosure.

FIGS. 11A-11C illustrate an example of three-dimensional (3D) evaluation using the detection system, in accordance with various embodiments of the present disclosure.

FIG. 12 is a schematic diagram illustrating an example of a computing system that can be utilized for breast cancer detection, in accordance with various embodiments of the present disclosure.

### DETAILED DESCRIPTION

Disclosed herein are various examples related to early breast cancer detection. As described above, it would be desirable to have a system and/or method for early breast cancer detection that are simple, accurate, and non-invasive, and that can be performed on a more frequent basis than a conventional annual examination. Disclosed herein are examples of such systems and methods. In one embodiment, a breast cancer detection system comprises an array of nodes that are integrated into a brassiere or other garment that can be applied to a patient's breasts. Each node comprises an electromagnetic emitter configured to emit electromagnetic (EM) radiation into the breast of the patient and a co-located temperature sensor configured to measure the temperature of the surface of the breast at the node location before and after irradiation. The temperature differential can be used to make a determination as to whether or not the breast tissue below the node contains a cancerous mass. In some embodiments, the determination of whether or not cancer is present is made using finite element analysis of the collected temperature data.

In the following disclosure, various specific embodiments are described. It is to be understood that those embodiments are example implementations of the disclosed inventions and that alternative embodiments are possible. Such alternative embodiments may include hybrid embodiments that comprise features from different disclosed embodiments. All such embodiments are intended to fall within the scope of this disclosure. Reference will now be made in detail to the description of the embodiments as illustrated in the drawings, wherein like reference numbers indicate like parts throughout the several views.

The disclosed systems and methods can use high-frequency electromagnetic waves, such as radio frequency (RF) waves, to excite breast tissue in a non-intrusive manner to help identify cancerous breast tissue, which responds differently than healthy breast tissue. This difference can then be mapped in terms of heat dissipation at the surface of the skin and the location, depth, and size of the cancerous tissue can be estimated. A three-dimensional thermal (3D) equivalent circuit can be used to identify the temperature distribution at different depths. The systems and methods therefore increase the sensitivity and accuracy of thermography. In addition, as the apparatus is relatively simple to use and inexpensive to produce, this form of breast cancer screening can be performed with greater frequency than traditional screenings and may potentially be performed by the patient herself at home. With greater scanning frequency, breast cancer may be detected earlier than through annual screenings alone. Furthermore, the systems and methods are suitable for use with a wide range of breast shapes and sizes without adversely affecting their accuracy. Finally, the patient is exposed to much less radiation during a scanning session than during a mammography examination. In fact, the patient is exposed to no more radiation than that emitted during a typical mobile phone call.

Cancerous tissue in breast, like most of the other cancer tissues, exhibit higher metabolic activities when compared to those of normal tissues surrounding them in lieu of the rapid pace of cell growth. These differences in energy consumptions lead to small but detectable surface temperature changes which can serve as the basis for breast cancer detection using thermal imaging. Although thermography has been studied for decades, the mechanisms of heat transfer between diseased and native tissue and the differences between the two have not been well described. Moreover, this method assumes that each breast has a particular thermographic pattern than does not change over time. This suggests that one can take a baseline and mark any significant changes observed in later images for future analysis. However, this assumption is not as reliable as once were thought. Human breast is a fairly complex organ that is made of various tissues and a labyrinthine of blood vessels. The particular thermographic pattern can experience significant changes depending on the physical conditions of different individuals. Also, if the patient has an asymmetrical body temperature, analysis of the thermographs could result in even higher false-negative and false-positive rates. It is for this reason that thermography alone is not recommended as a breast cancer screening method.

In this disclosure, the proposed method utilizes thermal imaging combined with external high frequency excitation. One advantage over the conventional technique is the independence of the symmetry and a particular thermographic pattern of a normal breast. Multiple excitation patch antennas can be included to serve as a source to slightly excite the breast simultaneously. The radio frequency waves radiated by the antennas establish an electric field inside the breast including the tumor tissue. Because of different electromagnetic and thermal properties between tumor and normal tissue, the losses caused by the rotation of molecular dipoles will heat up the tumorous tissue and normal tissue differently. Thus, the temperature increase reflected on the surface area with cancerous tissue underneath will be different from the healthy tissues.

The heat transfer in biological tissues and the relationship between tissue properties, such as metabolic rate and electromagnetic waves, has been investigated using comprehensive mathematical derivations. With the help of multi-physics simulation, the temperature and specific absorption rate (SAR) distributions that result from exposure to high-frequency electromagnetic fields have been analyzed. The results of that analysis have revealed that performing conventional thermography following a high-frequency excitation of breast tissue is a highly effective technique for detecting breast tissue abnormalities.

Heat transfer refers to the flow of thermal energy from one object to another object. Unlike heat transfer processes within a mechanical system or device, bio-heat transfer processes in living tissues are more complex because of the metabolic heat generation of the tissue and the exchange of thermal energy between flowing blood and the surrounding tissue. One can use the following partial differential to mathematically define this heat transfer:

$$\frac{\partial }{\partial x} ( k \frac{\partial T}{\partial x} ) + \frac{\partial }{\partial y} ( k \frac{\partial T}{\partial y} ) + \frac{\partial }{\partial z} ( k \frac{\partial T}{\partial z} ) + q = ρ {c}_{p} \frac{\partial T}{\partial t} , \qquad ( 1 )$$

where q, ρ, cₚ, k, and T denote the rate of energy generation per unit volume, density, specific heat, diffusion rate coefficients, and temperature, expressed in Cartesian coordinates (x, y, z), respectively.

Assuming constant thermal conductivity, there is no change in the amount of energy storage and the heat transfer can be one dimensional. In such a case, Equation (1) can be re-written as:

$$\frac{c {l}^{2} T}{\partial {x}^{2}} + \frac{q}{k} = 0 . \qquad ( 2 )$$

In the human body, both the metabolic heat generation and the exchange of thermal energy with the blood can be viewed as effects of thermal energy generation. Therefore, Equation (2) can be re-written as:

$$\frac{{d}^{2} T}{\partial {x}^{2}} + \frac{{q}_{m} + {q}_{b}}{k} = 0. \qquad ( 3 )$$

Where qₘ is the metabolic heat generation and q_{b} is the perfusion heat source that is responsible for energy exchange between the blood and the tissue.

According to Pennes' proposal, it can be assumed that, within any small volume of tissue, the blood flowing in the small capillaries enters at an arterial temperature Tₐ and exits at the local tissue temperature T. The perfusion term q_{b} can, therefore, be estimated as:

q_{b}=ωρ_{b}c_{b}(Tₐ−T), (4)

where ρ_{b}, c_{b}, and ω are the blood density, specific heat, and speed of blood perfusion, respectively. Substituting Equation (4) into Equation (3), one can obtain the following governing equation for heat transfer within breast tissue:

$$\frac{{d}^{2} T}{\partial {x}^{2}} + \frac{{q}_{m} + ω {ρ}_{b} {c}_{b} ( {T}_{a} - T )}{k} = 0. \qquad ( 5 )$$

To make the recorded temperature data meaningful, this group of antennas will keep their relative position from each other while rotating a small angle along the circumferential edge of the breast. After each rotation, a new set of excitations can be performed. In this way, the antenna group rotates and excites the breast multiple times to cover the entire surface of the breast. After each set of excitation and data recording, the breast is allowed to cool down to room temperature. Then a group of data can be obtained containing the temperature changing behavior before and after this small rotation. Through analyzing the temperature trend during the series of excitation, it can be determined if cancerous tissue is present and its approximate location can also be estimated.

Structure of Human Breast. In order to develop a reliable method for breast cancer detection, a firm understanding of the structure of human breast is important. A normal female human breast is a tear-shaped mammary gland that is designed to produce and secrete milk to feed infants. The breast comprises multiple layers of tissue with the majority of them being adipose (fat) and glandular tissue. Below the skin layer is the adipose tissue layer that extends all the way to the chest wall and spreads between the glandular tissues. The adipose layer envelops a network of milk ducts which originate from the glandular tissue called lobules, where milk is produced and stored.

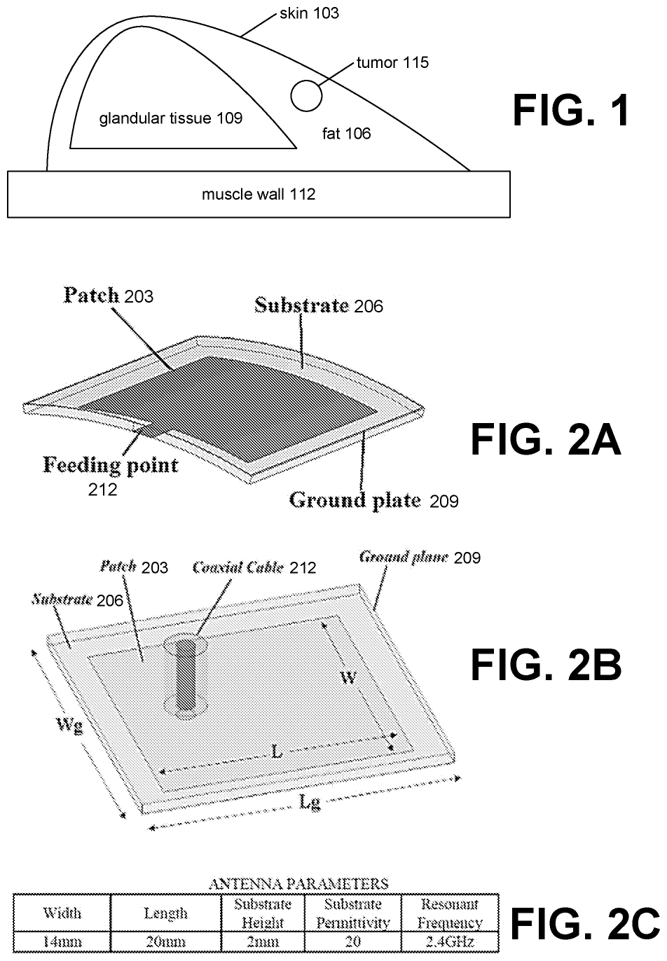

Multi-Physics Modeling of the Detection System. The breast model was directly drawn in High Frequency Simulation Software (HFSS). In order to have more accurate results and make the model as close as possible to reality, the general shape of the breast was designed into the shape of a tear, unlike most other studies where a hemisphere is employed. This FEA model, as illustrated in the cross-sectional view of FIG. 1, comprises four layers of the breast: skin 103, fat 106, gland 109, and muscle 112. FIG. 1 illustrates the 3D breast model and its cross-sectional view depicting the distribution of the different layers including a tumor 115. This model follows the general structure of the breast where the glandular tissue 109 in the center is surrounded by layers of adipose (fat) 106. The outermost layer is skin 103 whose thickness varies depending on locations. Everything is sitting on chest wall 112 that is made of muscle. Other less important parts of breast such as nipple and areola can be reasonably neglected. Since the method is designed for tumor detection, malignant tumor tissue 115 was also modeled. The electromagnetic properties of each layer along with their dependency on frequency was included in this model.

Patch antennas can be electromagnetic emitters chosen to be the external excitation source since they usually have a low profile and can be mounted on a flat surface. Patch antennas are also very practical at microwave frequencies, at which wavelengths are short enough so that the patches can be made conveniently small enough to be mounted near the surface of human breast. Generally, a basic patch antenna comprises a patch and a ground plane sitting on top of a dielectric substrate.

FIG. 2A illustrates an embodiment of an electromagnetic emitter that can be used in a breast cancer detection system. As shown in FIG. 2A, the emitter is configured as a patch antenna that includes a patch element 203 that is provided on an inner surface of a substrate 206 (facing the patient, adjacent to the skin). On the opposite side of the substrate is a ground plane 209. Electrical signals can be delivered to the patch element 203 via a feedline 212 to enable the emission of electromagnetic radiation into the breast. The substrate 206 can be made of a flexible material, such as liquid crystalline polymers or partially crystalline aromatic polyesters based on 4-hydroxybenzoic acid and related monomers (e.g., Ticona Vectra® A950 LCP), that can easily conform to the shape of breast.

FIG. 2B illustrates an example of the patch antenna that was modeled in HFSS. The distance between the patch 203 and the ground plane 209 equals the height of the substrate 206, which determines the bandwidth. The resonant frequency of the patch antenna can be determined by the length (L) and width (W) of the patch element 203 as well as the length (Lg) and width (Wg) of the ground plane 209. The length and width of the patch 203 and ground plane 209 listed in the table of FIG. 2C was calculated by the following equations:

$$L = {L}_{e f f} - 2 Δ L , \qquad ( 6 )$$

$$Δ L = 0.412 h \frac{( {ε}_{e f f} + 0.3 ) ( \frac{W}{h} + 0.264 )}{( {ε}_{e f f} + 0.258 ) ( \frac{W}{h} + 0.8 )} , \qquad ( 7 )$$

$${L}_{e f f} = \frac{c}{2 f \sqrt{{ε}_{e f f}}} , \qquad ( 8 )$$

$${ε}_{e f f} = \frac{{ε}_{r} + 1}{2} + \frac{{ε}_{r} - 1}{2} ( \frac{1}{\sqrt{1 + \frac{12 h}{W}}} ) . \qquad ( 9 )$$

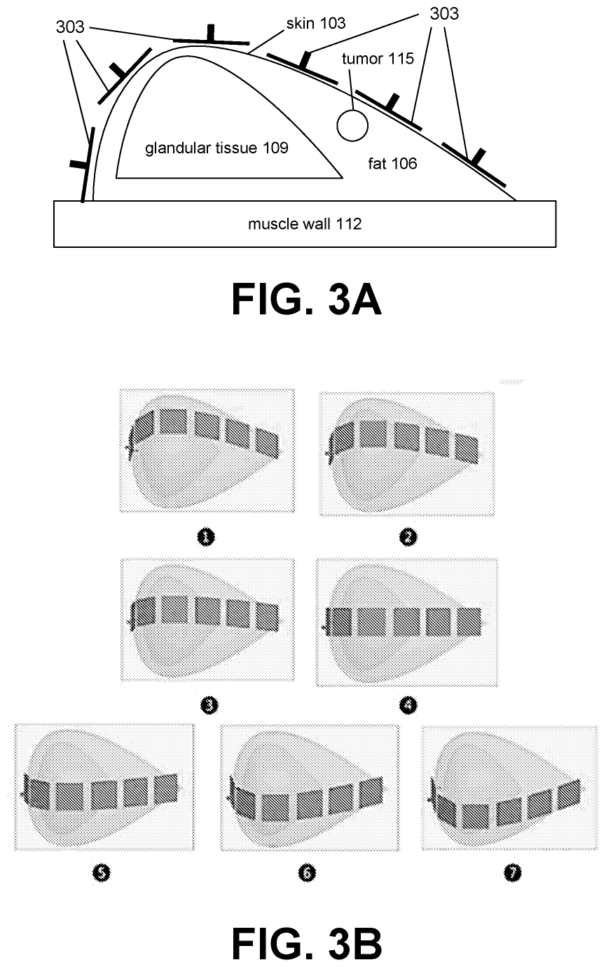

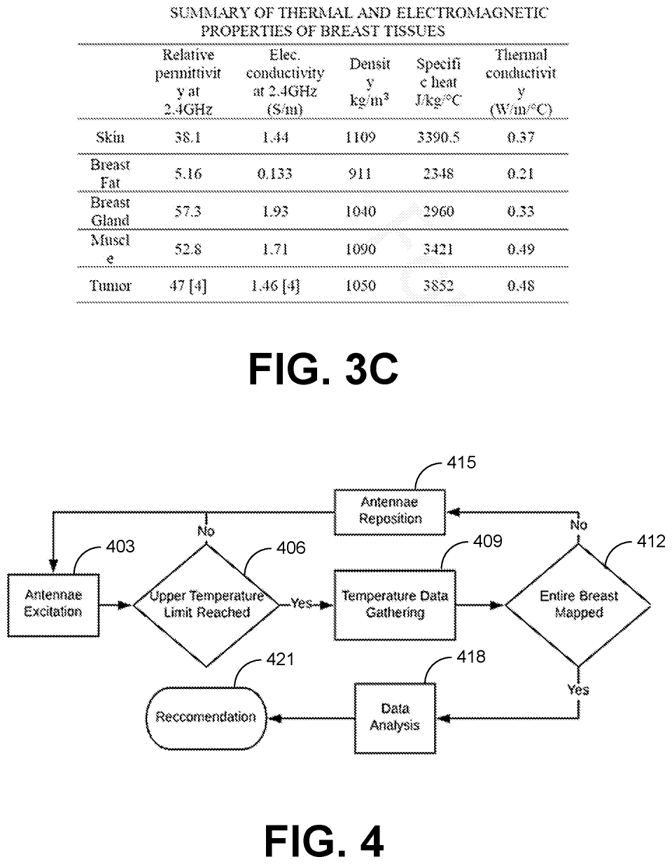

System Integration. This method employs a group of antennas to excite the breast multiple times. The arrangement of the antenna groups is shown in FIG. 3A where six antennas 303 were used to cover the longitudinal edge of the breast. The entire surface of the breast can be covered by rotating the antenna lines along the transversal direction as illustrated in FIG. 3B. Since the temperature distribution is utilized by this detection method, the electromagnetic simulation results were coupled to Ansys Mechanical to perform the thermal analysis. In this analysis ω, qₘ, ρ_{b} and c_{b} are the blood perfusion rate, metabolic heat generation rate, blood density and specific heat, respectively. The bio-heat transfer process with Pennes' bio-heat equation (5) was employed. The table of FIG. 3C summarizes both electromagnetic and thermal properties of different tissue layers used in this analysis.

FIG. 4 is a flow diagram illustrating an example of a screening procedure for breast cancer detection. In general, electromagnetic radiation, such as RF radiation having a frequency of approximately 3 kHz to 300 GHz, is sequentially emitted into the breast at discrete locations along the breast surface either using a single electromagnetic emitter that is moved from location to location, or using an array of electromagnetic emitters that is configured to extend across all or a portion of the breast surface.

When exposed to such radiation, any cancerous tissue within the breast tissue below the electromagnetic emitter generates heat that travels to the breast surface where the surface temperature can be measured by a temperature sensor such as, e.g., a thermistor, thermocouple or other appropriate thermal sensor. In some embodiments, each emitter is combined with co-located temperature sensor so as to form an integrated node. The difference between the breast surface temperature before and after the electromagnetic excitation provides an indication of the likelihood of cancerous tissue being present at that location.

To begin the scanning process, one or more electromagnetic emitters are positioned on the breast surface. At 403, electromagnetic radiation is then emitted into the breast tissue at a first location on the breast surface until an upper temperature limit is reached at 406, as measured by a temperature sensor. Once the temperature limit is reached at 406, the excitation is ceased and the temperature of the breast surface at that location is gathered and stored at 409. At this point, the before/after temperature differential and the thermal transient (i.e., the time rate of change in the temperature) at that location can be determined. This process can then be repeated for each of a plurality of discrete locations on the breast surface until temperature differentials are acquired at spaced locations across the entire surface of the breast. If the entire breast has not been mapped at 412, then the antennae can be repositioned at 415 and the excitation repeated at 403.

FIG. 3B illustrates a linear array of electronic emitters of the type shown in FIG. 3A. As is apparent from FIG. 3B, the linear array can be positioned at an initial location on the breast to acquire temperature differentials at multiple vertically spaced locations (7 locations in this example) across the breast (e.g., from the top of the breast to the bottom of the breast as shown in FIG. 3B). Once temperature differentials are obtained for each emitter location, the array can be moved (laterally in this example) along the surface of the breast and further temperature differentials can be acquired. This process can be repeated until temperature differentials have been obtained from discrete locations across the entire surface of the breast.

If the entire breast has been mapped at 412, then the acquired data can next be analyzed at 418, for example, using finite element analysis in which a precise heat transfer model is applied, and a determination can be made at 421 as to the presence (or absence) of cancerous tissue within the breast by considering several parameters, such as the heat distribution patterns (i.e., the distribution of the heat under the skin and within the breast tissue determined by measuring the temperature distribution on the surface and computing the heat distribution inside the breast using an electrothermal equivalent model), standard deviations (the standard deviations being used to identify the radical changes in the temperature on the surface. While average temperature changes tend to be small in magnitude, standard deviation, a second order statistical moment, identifies the areas with highest rate of change), and high temperature ratios (another numerical metric that considers the largest increase in the magnitude of the temperature after RF excitation).

Excitation time is an important variable in the above-described process as it controls the amount of electromagnetic energy that radiates into the breast. As the excitation time increases, so too does the amount of radiation that is emitted into the breast tissue, thus increasing the temperature of the entire breast tissue until an equilibrium state is reached. As noted above, excitation is radiated into the breast until a particular (or predefined) temperature is reached. In some embodiments, that temperature is the temperature at which the breast surface temperature has increased by 1° C.

The electromagnetic waves radiated by the emitters generate electromagnetic fields inside the breast, including any cancerous tissue. Due to the different electromagnetic and thermal properties of cancerous and healthy tissue, the loss by the rotation of molecular dipole affects the cancerous tissue and healthy tissue differently. Therefore, the temperature increase observed at locations on the breast surface where under which cancerous tissue lies will be larger than the healthy tissues.

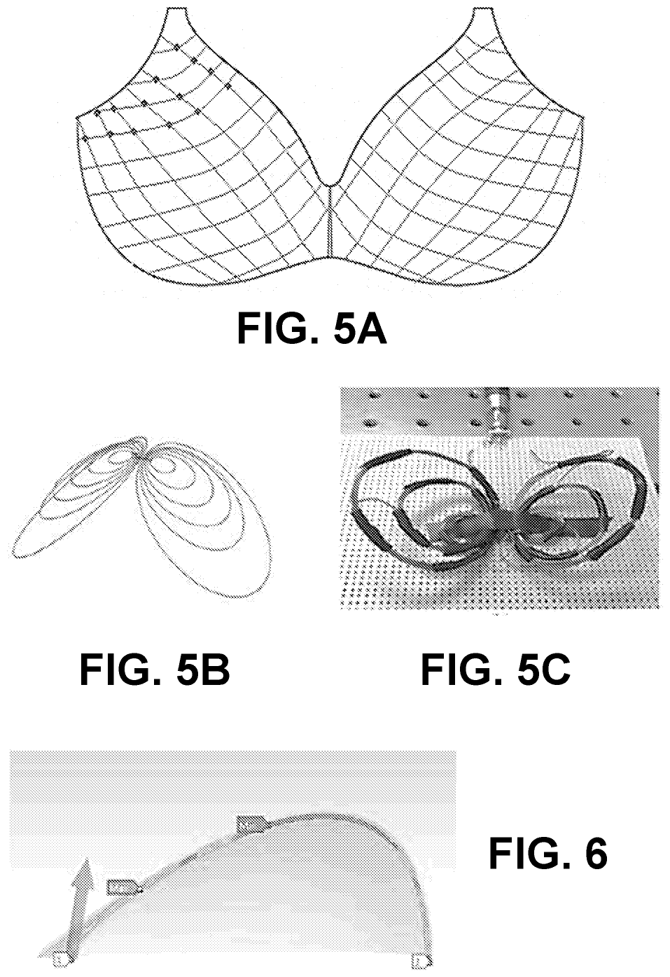

While repositioning an electromagnetic emitter or a linear array of electromagnetic emitters to different locations on the breast surface is feasible, the need for such repositioning can be eliminated through incorporation of two-dimensional arrays of emitters (arranged in matrices of rows and columns) into the breast cups of a brassiere, such as that illustrated in FIG. 5A. As shown in FIG. 5A, the array of emitters (identified by dots) form a generally orthogonal grid across the inside of each breast cup so as to be configured to contact the breast surface when the brassiere is donned. As noted above, each emitter can be accompanied by a co-located temperature sensor, in which case the dots in FIG. 5A identify integrated nodes, each comprising an emitter and a temperature sensor.

In some embodiments electrical signals can be sequentially transmitted to each node along electrical conductors also integrated into the brassiere using a control unit (not shown). Such a control unit can comprise a microcontroller, a power source (e.g., battery), and a multiplexer that can be used to individually address each node. When provided, the control unit can be integrated into the brassiere, for example, attached to a back strap of the brassiere. In alternative embodiments, each electromagnetic emitter can be simultaneously excited. In such a case, the multiplexer and the various conductors required to independently address the emitters can be omitted.

While patch antennas have been identified as possible electromagnetic emitters, it is noted that other configurations are possible. FIGS. 5B and 5C illustrate one such example. As shown in FIG. 5B, an electromagnetic emitter can be configured as a multiple figure-eight element. Such an emitter element comprises multiple figure-eight elements, each comprising two generally circular loops whose peripheries are coupled together at a center point of the element. The multiple figure-eight element includes figure-eight elements of different sizes (i.e., the loops have different dimensions, such as diameters) that overlap each other at their respective centers. Such a configuration increases the depth of penetration of the emitted electromagnetic radiation and, therefore, may result in deeper cancerous masses being excited and detected. FIG. 5C is an image of a fabricated electromagnetic emitter comprising figure-eight elements.

### Simulation Results

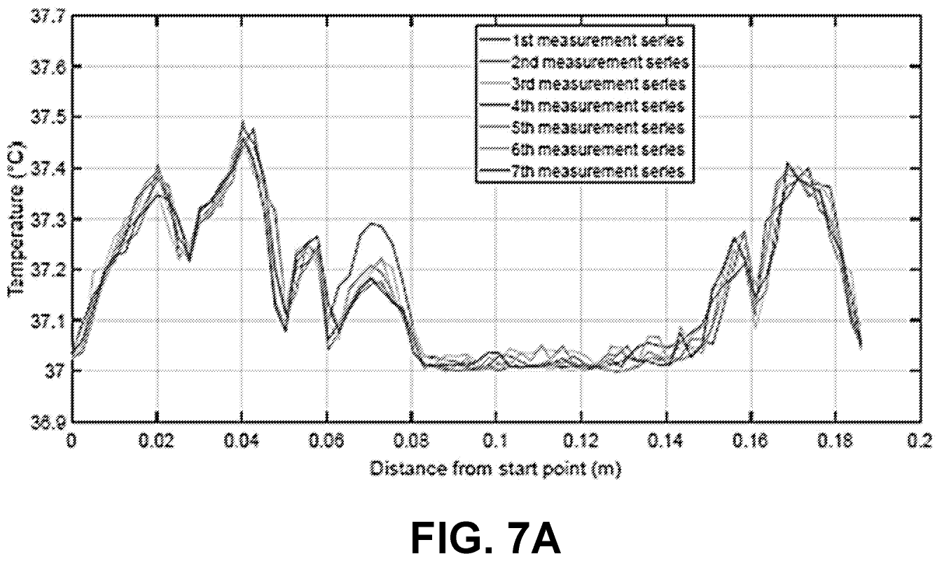

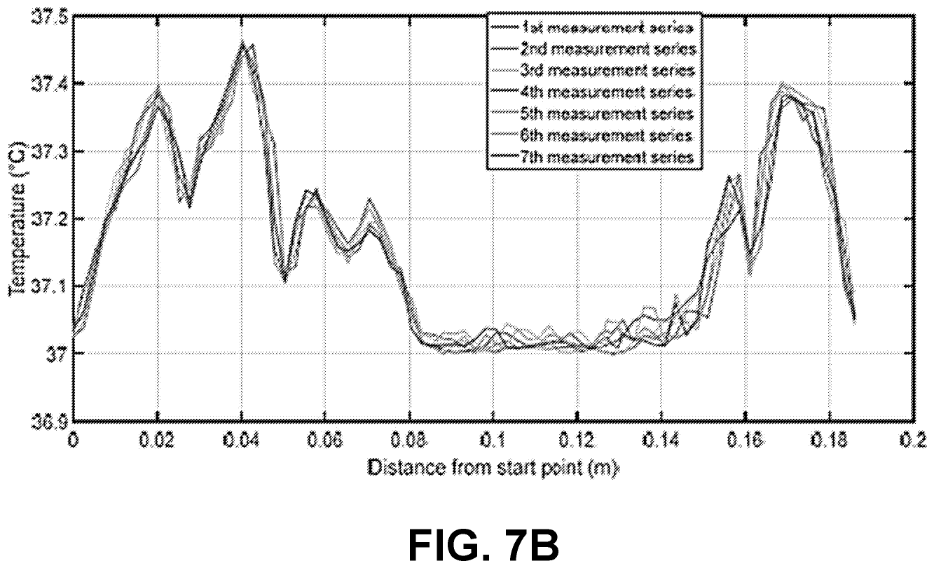

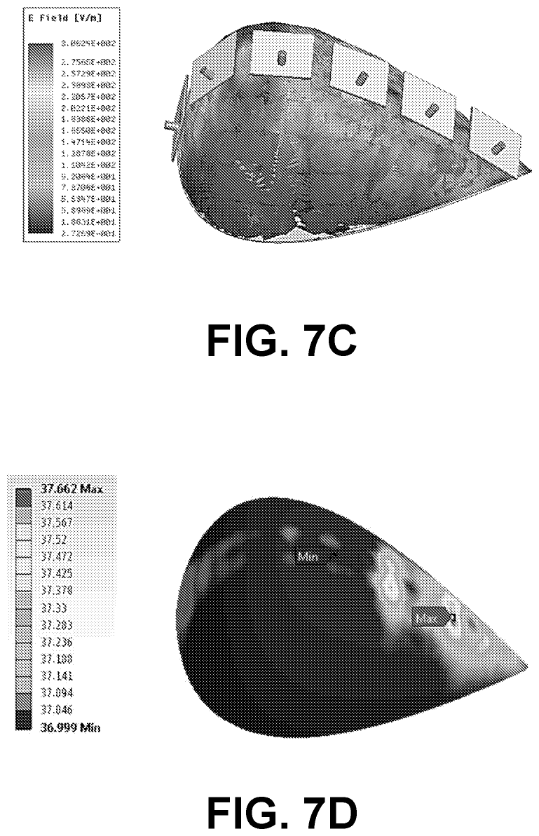

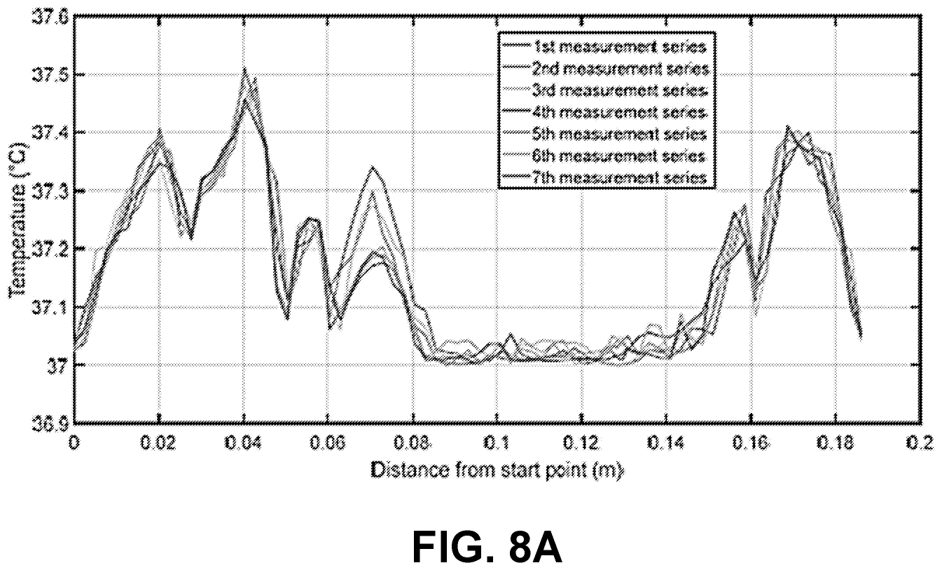

In order to make the results to be more reasonable in real-life application, the specific absorption rate (SAR) and maximum temperature increase was checked to make sure they are within safety regulation. In the post processing procedure after each simulation, the temperature along a circumferential line located right below the rotating antennas on the skin surface was measured. The measurement starting and end points are illustrated in FIG. 6. The tumor was modeled into a location in the breast fat tissue as shown in FIGS. 1 and 3A. Seven simulations corresponding to seven different locations of the antenna group have been performed to plot the surface temperature trend. The results were plotted in FIG. 7A and also compared with a breast without embedded tumor in FIG. 7B. The horizontal axis is the distance from the measurement starting point while the vertical axis is temperature. The seven measurement series correspond to seven excitations with different antenna group locations. Several temperature hot spots are shown in the plot corresponding to each antenna location. It can be seen that the temperature distribution along the antenna line before and after rotation has a similar pattern, with similar peaks and troughs except at one location: at approximately 0.07 m. FIGS. 7C and 7D illustrate the electric field distribution and its corresponding temperature distribution due to the established field, respectively.

When compared with FIGS. 1 and 3A, it can be seen that under the skin of this location is approximately where the tumor 115 is located. The reason for this difference in temperature can be attributed to the sudden change of material properties as the antennas rotate. In areas where there is no tumor embedded, the tissue properties are consistent. As antenna rotates, the established electric field under the antenna is approximately the same, thus resulting in the same temperature rise. While in an area where a tumor is embedded, the EM waves experience a sudden change in material properties between two consecutive excitations, thus the induced electric field would be different, resulting in a different temperature rise. Different from conventional thermography, the use of an external excitation source reveals the changing trend of surface temperatures between healthy and malignant areas, therefore this method does not require comparison between particular thermographs.

The size of a cancerous tumor is an important indicator of the breast cancer stage. As one may expect, the malignancy of breast cancer increases with the increase in tumor size. FIGS. 7A, 7B and 8A are graphs that show the temperature distributions for three breast models after 30-second electromagnetic excitations at each location. FIG. 7B shows data for a breast model having no tumor, FIG. 7A shows data for a breast model having a 10 mm tumor, and FIG. 8A shows data for a breast model having a 15 mm tumor. The temperature differential at points 0.06 m to 0.08 m in each graph are easily distinguishable from each other because the breast tissue that contained a tumor resulted in a greater temperature increase as compared to breast tissue without a tumor. Also, the larger tumor generated a greater temperature increase than the smaller tumor. It was also confirmed that moving the location of the tumor resulted in a similar temperature increase at the new location. This is shown in FIG. 8B for a series in which the 15 mm tumor was embedded in a different location within the breast model.

### Experimental Verification

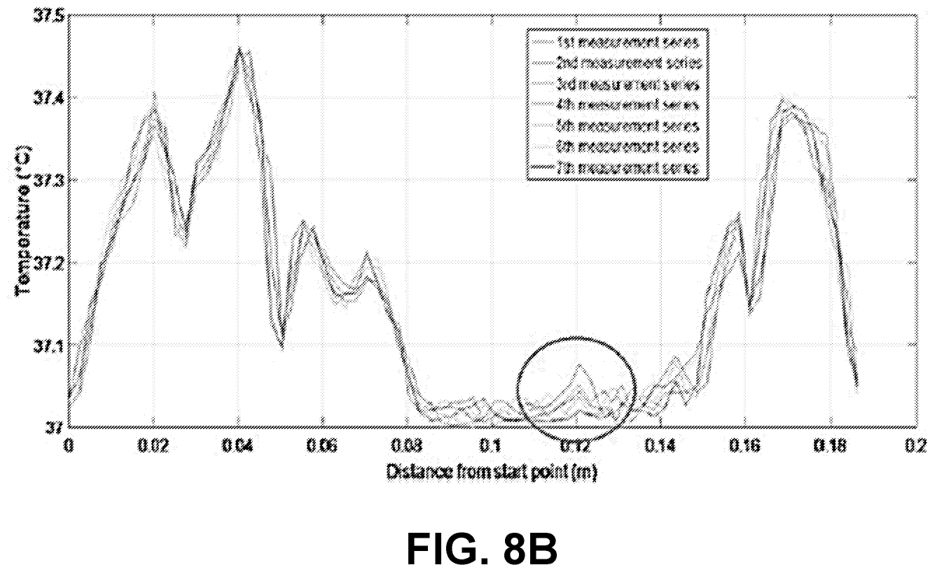

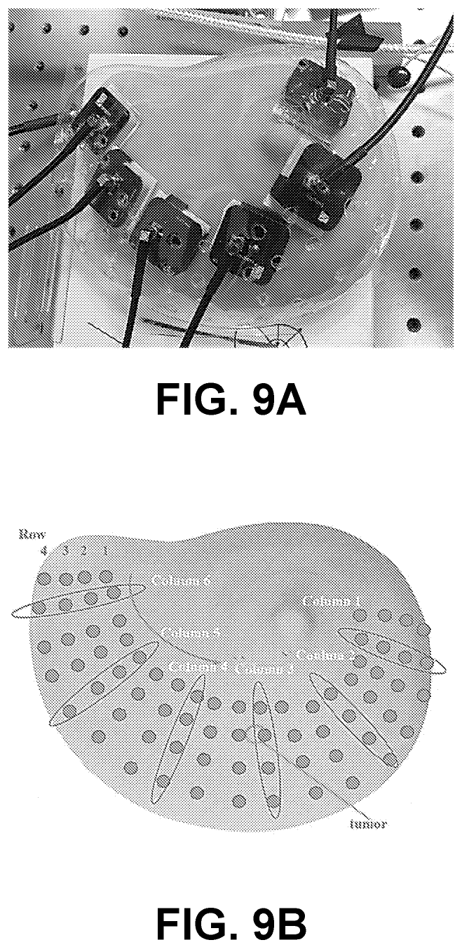

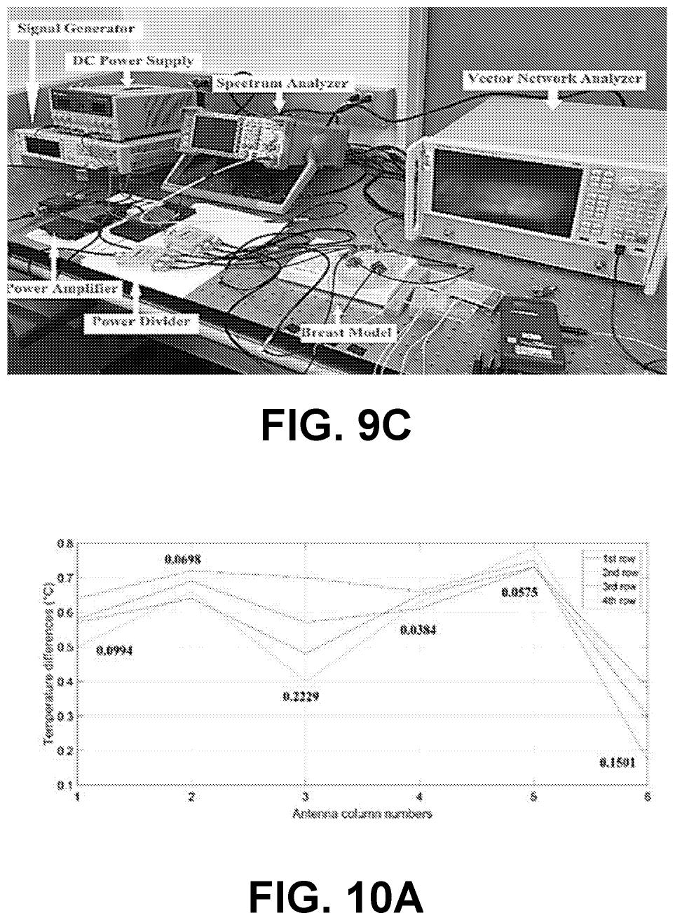

Experiments were performed to test the disclosed breast cancer detection techniques described above. A human breast model (phantom) was obtained, and the multi-physics simulation process of the novel breast cancer detection technique based on a combination of high frequency excitation and thermal analysis was applied to the model. FIG. 9A is an image of the breast model with multiple patch antennae applied thereto. FIG. 9B identifies the various locations across the surface of the model at which temperature differentials were acquired. The FEA model and the detection technique was tested using a prototype system and the experimental measurements were compared to the simulation results. The detection system setup including the breast model is shown in FIG. 9C. The test setup also includes a high frequency signal generator, a DC power supply, a spectrum analyzer, two power amplifiers, three power dividers, a vector network analyzer, several thermistors, several patch antennas and a digital thermometer.

FIG. 9B demonstrates the patch antenna distribution on the breast model. The tumor location was indicated by the circle. A plastic shell was used to support the antennas. It also helps in fixing the relative locations between antennas and the breast as well as locations between different antennas. The solid circles indicate where the holes are on the plastic shell. The antennas were arranged in such a way that there was one side in parallel with the other sides. On the plastic shell, there were four rows of holes shown in the circles (the rows are along the direction of the arrow in FIG. 9B) covering the majority area of the breast surface along the radial direction.

During the first round of experiment, all the antennas were fixed into the first row of holes and excited using the signal generator. After the temperature under each antenna was measured, the entire antenna group was moved to the next row. In this way, four sets of excitations were performed with the antenna group in those four rows. In order to cover as much area as possible, the antenna group can also move along the tangential direction. There are another two holes within the same row for each antenna to cover the tangential direction of the breast. As a result, a complete test contains 72 holes and 12 sets of excitations in total. Both initial and final temperatures were measured. After measurement of temperature under one antenna, the model was allowed to cool down to room temperature before another round of excitation. Although the room temperature fluctuates, the excitation period is so short (20-45 s) that the change in room temperature can be reasonably disregarded.

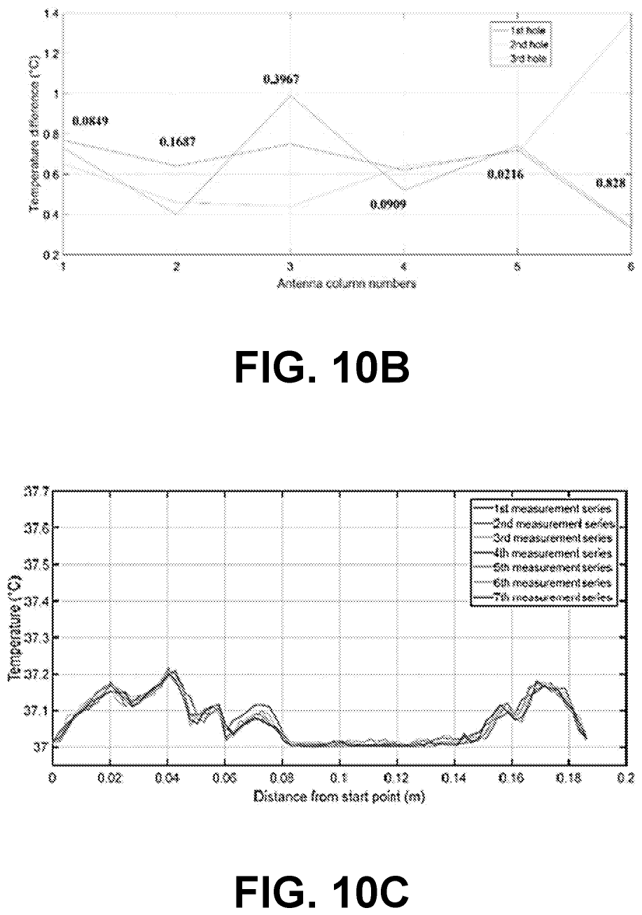

The experiment was first performed by placing the antennas in the location indicated by the 24 solid circles (six encircled groups of four). The excitation lasted for 30 s. Their initial and final temperature were measured and compared. FIG. 10A illustrates the temperature difference for different rows and columns. As can be seen in FIG. 10A, although the initial temperature is different for each point, the induced temperature increase in a given column is approximately the same for most cases except in column 3 at row 2 where the temperature increase is apparently higher than the surrounding points. This difference in temperature increase is due to the presence of an abnormal tissue, in which the established electric field is different from those in the surroundings areas.

As shown in FIG. 9B, there are two additional holes to place antennas for each row and column in order to cover the tangential direction of the breast. In the second test, the antenna group was moved to the second row and the antennas were moved along the tangential direction on the breast surface. The excitation time was also 30 s. FIG. 10B illustrates the temperature difference between initial and final temperature for different holes along the 2nd row at different columns. As shown in FIG. 10B, a fairly constant temperature increase exists among different columns but the variations within the same column is larger especially for column 3 and 6. The larger variation among different holes in column 3 is consistent with simulation results because the tumor location is between the 2nd and 3rd hole in column 3 resulting in a larger variation in temperature difference between the 2nd hole location and 1st hole location as well as between the 3rd hole location and the 1st hole location. The large variation in the 6th column is due to the existence of a fibrocystic mass in the corresponding location in the breast model.

FIG. 10C is a graph of seven repeated experiments in which each line contains data from one data series, i.e., a series of measured temperatures at a specific location on the surface with its distance from a reference point linearly increased, captured using 10-second electromagnetic excitations at each location.

It is noted that a three-dimensional distribution of temperature within the breast can also be obtained for the purpose of detecting breast cancer. Access to the temperature distribution inside the breast provides higher fidelity of detection of unusual sources of heat inside the breast and, therefore, increases the accuracy with which the location of potential tumors can be identified.

In some embodiments, a three-dimensional detection can be performed that is based on an electro-thermal equivalent circuit for various slabs of the breast (e.g., 10 mm thick transverse slices of the breast) based on their typical bio-thermal properties. A software program configured to perform the three-dimensional analysis can start with an approximate location of a tumor obtained from the two-dimensional analysis of the surface temperature described above. The thermal equivalent circuit can then be used to solve a reverse electro-thermal problem to obtain estimates of the temperatures at various depths within the breast. The accuracy of the technique can be confirmed by comparing the temperature distribution on all sides of the breast surface.

Distribution of the material and properties (i.e., permittivity, permeability, and conductivity) is provided for the phantom (for gland, fat and tumor). However, these quantities can be computed from analyzing the reflection and absorption coefficients. The Nicholson Ross Weir (NRVV) derivation is an analytic method that calculates permittivity and permeability from measure S11 and S21 parameters. Consider a system of air/sample/air, then the incident wave travels and a first partial reflection on the air-sample interface occurs. The remaining portion of the signal continues to travel through the sample and on the second air-sample interface part of the signal transmits and the other part reflects back and travels through the sample toward the first air-sample interface.

After simplification of expressions that include all terms of multiple reflections and transmissions, the final expression for the total reflection parameter, S₁₁, and the total transmission parameter, S₂₁, can be given as:

$${S}_{11} = \frac{{G}_{1} \cdot ( 1 - {x}^{2} )}{1 - {G}_{1}^{2} \cdot {x}^{2}} \qquad ( 10 )$$

$${S}_{21} = \frac{z \cdot ( 1 - {G}_{1}^{2} )}{1 - {G}_{1}^{2} \cdot {x}^{2}} \qquad ( 11 )$$

$${x}^{2} = {e}^{- 2 \cdot j \cdot γ \cdot d} \qquad ( 12 )$$

$${G}_{1} = \frac{Z - 1}{Z + 1} , Z = \sqrt{\frac{μ}{ϵ}} \qquad ( 13 )$$

where x is an unknown variable that depends on the propagation constant γ, d is the sample thickness, G₁ is the first partial reflection on the air-sample interface, and Z is the characteristic impedance of the material. The relation of the propagation constant γ with ϵ and μ of the material is given as:

$$γ = \frac{j \cdot 2 \cdot π \cdot \sqrt{μ ϵ}}{λ} , γ = \frac{1}{d} \cdot log \frac{1}{x} \qquad ( 14 )$$

where λ is a free space wavelength and d is the material slab thickness. Nₘ is the material's refractive index and can be expressed in terms of permittivity and permeability as:

Nₘ=\sqrt{μ·ϵ}. (15)

By combining Equation (6), the permittivity and permeability can be expressed as:

$${N}_{m} = \frac{- j \cdot λ \cdot γ}{2 \cdot π} , ϵ = \frac{{N}_{m}}{Z} , μ = {N}_{m} \cdot Z . \qquad ( 16 )$$

This method can give much higher fidelity to the thermography technique and will be the equivalent of a thermal MRI, which can prove the presence and provide the location of tumors with much higher precision.

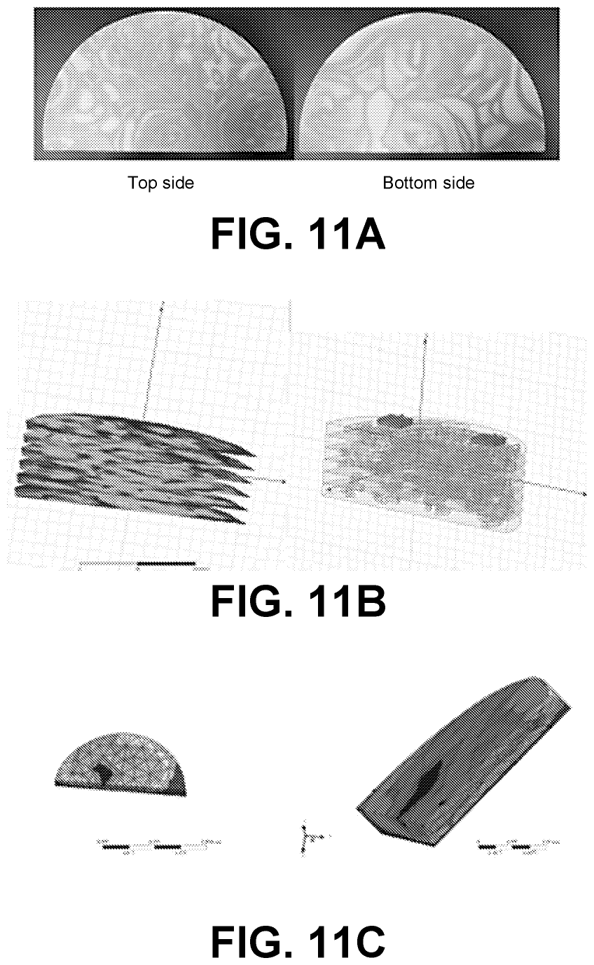

FIG. 11A shows an example of a multi-slab phantom of a breast that was exposed to RF excitation. FIG. 11B shows a sample result for the electric field distribution and FIG. 11C shows an example of a three-dimensional temperature distribution of the multi-slab phantom.

The combination of high-frequency electromagnetic fields and thermography described in this disclosure provides a simple, accurate, and non-invasive approach to breast cancer detection that can be performed with greater frequency than annual mammograms. If prescribed to a patient for take-home use, the apparatus could be used with much higher frequency, for example, on a weekly or monthly basis, if desired. In some embodiments, the data that is collected by the control unit can be transmitted to a patient device, such as a smart phone, and then further transmitted to the patient's physician for analysis, review, and recordation. Accordingly, cancer can be detected early and, if desired, further testing, such as mammography, can be performed to confirm the cancer's presence.

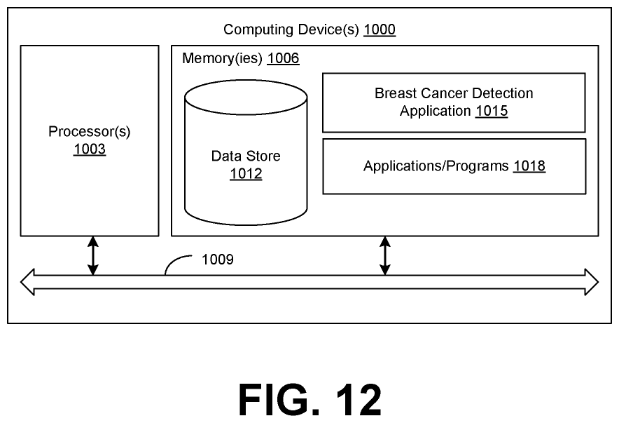

Referring next to FIG. 12, shown is a schematic block diagram of a computing device 1000 such as, e.g., the control unit of the system. In some embodiments, among others, the computing device 1000 may represent a mobile device (e.g. a smartphone, tablet, computer, etc.). Each computing device 1000 includes at least one processor circuit, for example, having a processor 1003 and a memory 1006, both of which are coupled to a local interface 1009. To this end, each computing device 1000 may comprise, for example, at least one server computer or like device, which can be utilized in a cloud based environment. The local interface 1009 may comprise, for example, a data bus with an accompanying address/control bus or other bus structure as can be appreciated.

In some embodiments, the computing device 1000 can include one or more network interfaces. The network interface may comprise, for example, a wireless transmitter, a wireless transceiver, and/or a wireless receiver (e.g., Bluetooth®, Wi-Fi, Ethernet, etc.). The network interface can communicate with a remote computing device using an appropriate communications protocol. As one skilled in the art can appreciate, other wireless protocols may be used in the various embodiments of the present disclosure.

Stored in the memory 1006 are both data and several components that are executable by the processor 1003. In particular, stored in the memory 1006 and executable by the processor 1003 are at least one breast cancer detection application 1015 and potentially other applications and/or programs 1018. Also stored in the memory 1006 may be a data store 1012 and other data. In addition, an operating system may be stored in the memory 1006 and executable by the processor 1003.

It is understood that there may be other applications that are stored in the memory 1006 and are executable by the processor 1003 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 1006 and are executable by the processor 1003. In this respect, the term “executable” means a program or application file that is in a form that can ultimately be run by the processor 1003. Examples of executable programs may be, for example, a compiled program that can be translated into machine code in a format that can be loaded into a random access portion of the memory 1006 and run by the processor 1003, source code that may be expressed in proper format such as object code that is capable of being loaded into a random access portion of the memory 1006 and executed by the processor 1003, or source code that may be interpreted by another executable program to generate instructions in a random access portion of the memory 1006 to be executed by the processor 1003, etc. An executable program may be stored in any portion or component of the memory 1006 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1006 is defined herein as including both volatile and nonvolatile memory and data storage components. Volatile components are those that do not retain data values upon loss of power. Nonvolatile components are those that retain data upon a loss of power. Thus, the memory 1006 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores and the memory 1006 may represent multiple memories 1006 that operate in parallel processing circuits, respectively, such as multicore systems, FPGAs, GPUs, GPGPUs, spatially distributed computing systems (e.g., connected via the cloud and/or Internet). In such a case, the local interface 1009 may be an appropriate network that facilitates communication between any two of the multiple processors 1003, between any processor 1003 and any of the memories 1006, or between any two of the memories 1006, etc. The local interface 1009 may comprise additional systems designed to coordinate this communication, including, for example, performing load balancing. The processor 1003 may be of electrical or of some other available construction.

Although the breast cancer detection application 1015 and other applications/programs 1018, described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the breast cancer detection application 1015 and other applications/programs 1018, that comprises software or code can be embodied in any non-transitory computer-readable medium for use by or in connection with an instruction execution system such as, for example, a processor 1003 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the breast cancer detection application 1015 and other applications/programs 1018, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 1000, or in multiple computing devices in the same computing environment. Additionally, it is understood that terms such as “application,” “service,” “system,” “engine,” “module,” and so on may be interchangeable and are not intended to be limiting.

A novel breast cancer detection technique utilizing a combination HF excitation and surface temperature analysis has been disclosed. The structure of a human breast was accurately modeled using high frequency FEA approach. Subsequently the detection system was modeled and shown to be effective. 3D analysis can provide additional information about the detected cancer. Consequently, a testbed was developed so that the simulation results and the feasibility of the detection method could be experimentally verified.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A method for performing a breast examination to detect cancer, the method comprising:
measuring a first temperature of a discrete location on the surface of the breast;
emitting electromagnetic radiation into the breast at the discrete location to excite any cancerous tissue below the surface and cause the cancerous tissue to generate heat;
enabling the generated heat to reach the surface of the breast;
measuring a second temperature of the discrete location on the surface of the breast;
determining a temperature differential between the first and second temperatures; and
determining whether or not cancerous tissue exists within the breast at the discrete location based upon the temperature differential.

2. The method of claim 1, further comprising determining a temperature differential in the same manner for multiple discrete locations on the surface of the breast.

3. The method of claim 2, wherein the temperature differential is determined by sequentially exciting individual elements of an electromagnetic emitter array positioned over the breast.

4. The method of claim 3, comprising determining a three-dimensional temperature distribution in the breast based on an electro-thermal equivalent circuit of the breast, the electro-thermal equivalent circuit based upon bio-thermal tissue properties of the breast.

5. The method of claim 4, wherein a location of the cancerous tissue is identified based upon the three-dimensional temperature distribution.

6. The method of claim 1, wherein emitting electromagnetic radiation comprises emitting radio frequency radiation.

7. The method of claim 6, wherein the radio frequency radiation is in a frequency range from about 3 kHz to about 300 GHz.

8. The method of claim 1, wherein determining whether or not cancerous tissue exists within the breast at the discrete location comprises performing finite element analysis.

9. The method of claim 1, comprises:
measuring a first temperature of a second discrete location on the surface of the breast;
emitting the electromagnetic radiation into the breast at the second discrete location to excite any cancerous tissue below the surface and cause the cancerous tissue to generate heat; and
measuring a second temperature of the second discrete location on the surface of the breast.

10. The method of claim 9, wherein the electromagnetic radiation is emitted at the two discrete locations by two different electromagnetic emitters positioned over the breast.

11. The method of claim 10, wherein the two different electromagnetic emitters are integrated in a garment worn over the breast.

12. The method of claim 9, wherein the electromagnetic radiation is sequentially emitted at the two discrete locations by an electromagnetic emitter that is repositioned from the discrete location to the second discrete location.

13. A system for performing a breast examination to detect cancer, the system comprising:
a plurality of electromagnetic emitters provided within an array;
a plurality of temperature sensors, one temperature sensor co-located with each electromagnetic emitter;
a garment that supports the emitters and temperature sensors configured for application to a breast of a patient; and
a control unit configured to activate the electromagnetic emitters for the purpose of emitting electromagnetic radiation into the breast and to receive temperature measurements taken by the temperature sensors.

14. The system of claim 13, wherein the electromagnetic emitters comprise patch antennas.

15. The system of claim 13, wherein the electromagnetic emitters comprise multiple figure-eight elements.

16. The system of claim 13, wherein the temperature sensors comprise thermistors.

17. The system of claim 13, wherein the control unit obtains a first temperature measurement prior to emitting the electromagnetic radiation into the breast by one of the plurality of electromagnetic emitters and a second temperature measurement after emitting the electromagnetic radiation into the breast, the temperature measurements obtained by the temperature sensor co-located with the one electromagnetic emitter.

18. The system of claim 17, wherein presence of cancerous tissue is determined based upon a temperature differential determined from the first and second temperature measurements.

19. The system of claim 17, wherein temperature differentials are determined by sequentially exciting individual elements of the plurality of electromagnetic emitters positioned over the breast.

20. The system of claim 13, wherein the plurality of electromagnetic emitters and the plurality of temperature sensors are configured for application to both breasts of the patient.
